(19) United States
(12) Patent Application Publication
(10) Pub. No.: US 2024/0350150 A1
(43) Pub. Date: Oct. 24, 2024

(54) OFF-AXIS ROTARY TOOL

(71) Applicant: Fidelis Partners, LLC, Cheyenne, WY (US)

(72) Inventor: Christopher PEDICINI, Brentwood, TN (US)

(73) Assignee: Fidelis Partners, LLC, Cheyenne, WY (US)

(21) Appl. No.: 18/640,604

(22) Filed: Apr. 19, 2024

Related U.S. Application Data
(60) Provisional application No. 63/460,472, filed on Apr. 19, 2023.

(51) Int. Cl. A61B 17/16

(52) U.S. Cl. CPC A61B 17/1666; A61B 17/1615; A61B 17/1628

(57) ABSTRACT

An off-axis rotary tool may include a motor drive and a cutting head having teeth. During an operational cycle of the off-axis rotary tool, the cutting head may simultaneously precess about a precession axis and rotate about a rotation axis.

### CROSS-REFERENCE TO RELATED APPLICATION

This application claims the benefit of U.S. Provisional Application No. 63/460,472, filed Apr. 19, 2023, which is incorporated herein by reference in its entirety.

### BACKGROUND

Rotary tools, such as reamers, are cutting tools that may be used in surgery to enlarge or shape holes or cavities in bone or other hard tissues. For example, reamers are commonly used in orthopedic surgery to prepare bone surfaces for insertion of implants or prosthetic components, such as joint replacements.

### SUMMARY

Some implementations described herein relate to an off-axis rotary tool. The off-axis rotary tool includes a motor drive a cutting head, coupled to the motor drive, including cutting teeth. During an operational cycle of the off-axis rotary tool, the cutting head simultaneously precesses about a precession axis and rotates about a rotation axis.

Some implementations described herein relate to a method for operating an off-axis rotary tool including a motor drive and a cutting head, coupled to the motor drive, including cutting teeth. The method includes causing, via the motor drive, the cutting head to simultaneously precess about a precession axis and rotate about a rotation axis.

Some implementations described herein relate to an off-axis reamer for orthopedic surgery. The off-axis reamer includes a motor drive and a reamer head, coupled to the motor drive, including cutting teeth. During an operational cycle of the off-axis reamer, the reamer head simultaneously precesses about a precession axis and rotates about a rotation axis.

### BRIEF DESCRIPTION OF THE DRAWINGS

FIGS. 1A-1F are diagrams of an example associated with an improved rotary tool, in accordance with some embodiments of the present disclosure.

FIG. 2 is a flowchart of an example process associated with an improved rotary tool, in accordance with some embodiments of the present disclosure.

### DETAILED DESCRIPTION OF EXAMPLE EMBODIMENTS

The following detailed description of example implementations refers to the accompanying drawings. The same reference numbers in different drawings may identify the same or similar elements.

Robotic assistance (e.g., via a surgical robot and/or via robotic automation, among other examples) may be used in a surgical procedure. However, while robotic assistance has been effectively used for surgeries having low force and low energy requirements (e.g., laparoscopic procedures, among other examples), robotic assistance faces challenges and problems when used for surgeries having high force and high energy demands (e.g., orthopedic procedures, among other examples).

To operate during a surgical procedure, a typical surgical robot identifies (e.g., via a registration technique) a bone geometry of a surgical site (e.g., to accurately navigate, guide, and manipulate an end effector, coupled to the typical surgical robot, through the surgical site). However, in some cases, typical surgical robots cannot handle a magnitude (e.g., a relatively high magnitude) of reactionary forces that result from typical large bone surgical tools (e.g., saws and/or reamers) and cannot provide a sufficient linear force (e.g., a linear support force or a linear push force) to advance the typical large bone surgical tools medially (e.g., during a reaming procedure).

As an example, during orthopedic surgeries, a typical surgical power tool may be used as the end effector, which produces a significant amount of torque (e.g., when the end effector is a reamer) or shock (e.g., when the end effector is a surgical impacting tool). This torque or shock not only can causes the typical surgical robot to lose its registration, but the torque or shock can also damage one or more components of the typical surgical robot (e.g., intricate machinery and/or components of the typical surgical robot). As a result, the typical surgical robot requires a surgeon to be present during surgical procedures to not only handle the torque and/or the shock, but also to apply a sufficient linear force to drive the typical surgical power tool (e.g., the reamer) medially into a cavity (e.g., a cavity of an acetabulum), which is also referred to as pushing a medial wall of the acetabulum back (e.g., backwards).

With regard to typical reamers in particular, it is understood that typical reamers may be used in surgery to prepare cavities in the bones which are intended to receive implants. These typical reamers generally have a hollow cutting head, which may be conical. This hollow cutting head is often fixed to an end of a rigid rod. Typically, a surgeon, or a surgical robot, starts with an undersized reamer cup to drive the medial wall of the acetabulum back.

For example, if the surgeon templates a size 52-millimeter (mm) cup implant for the acetabulum, the surgeon may start with a 47 mm reamer cup to drive the medial wall of the acetabulum backwards (e.g., between a range of 5 mm to 10 mm. This undersized reamer cup requires a large linear force (e.g., a large linear support force or a large linear push force) from the surgeon, or the robot, to advance the undersized reamer cup medially in the acetabulum. This linear force can be in excess of 60 pounds (lbs), which the typical surgical robot cannot provide. As a result, the surgeon is required to be present during reaming of the acetabulum to provide the reamer with the required linear force to drive the medial wall of the acetabulum backwards while the surgical robot attempts to guide a trajectory of the reamer. Accordingly, a need exists for improving operation of a surgical tool by a surgical robot and for a more efficient reamer (e.g., a reamer that requires less linear force to drive the medial wall of the acetabulum backwards).

Accordingly, some implementations described herein may provide a solution to high reactionary forces that occur from the use of orthopedic surgical tools. In this way, the reactionary forces upon a surgical robot and/or a surgeon are reduced, allowing better control of the orthopedic surgical tool (e.g., positioning thereof) during surgery. In addition to reducing the reactionary forces, some implementations described herein mechanically provide all, or a significant portion of, linear forces required to complete surgeries (e.g., orthopedic surgeries). In this way, the surgeon and/or the surgical robot only have to guide the orthopedic surgical tools while imparting minimal force onto the orthopedic surgical tools.

Additionally, some implementations described herein enable an off-axis rotary tool (e.g., an off-axis reamer) to create a cavity that does not contain excess material while also significantly reducing an amount of force required for cutting during an orthopedic procedure (e.g., by significantly reducing a frictional load associated with the surgical process), as described in more detail elsewhere herein. Furthermore, some implementations described herein enable the off-axis rotary tool to provide sufficient linear force to move (or advance) the off-axis rotary tool medially, as described in more detail elsewhere herein.

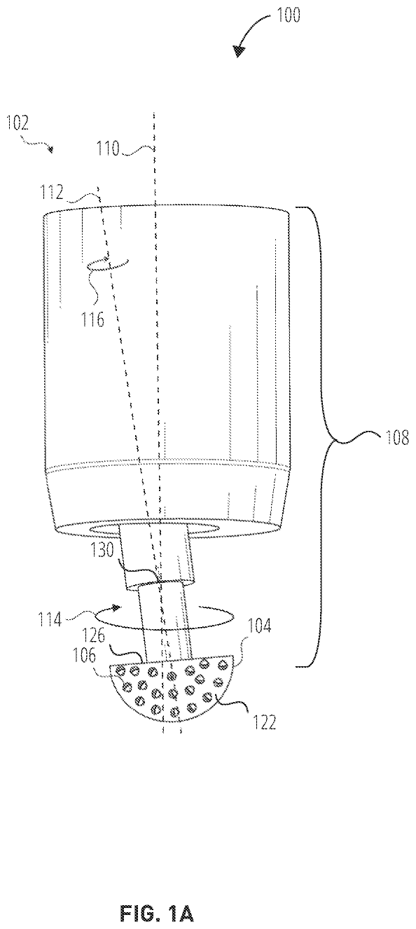

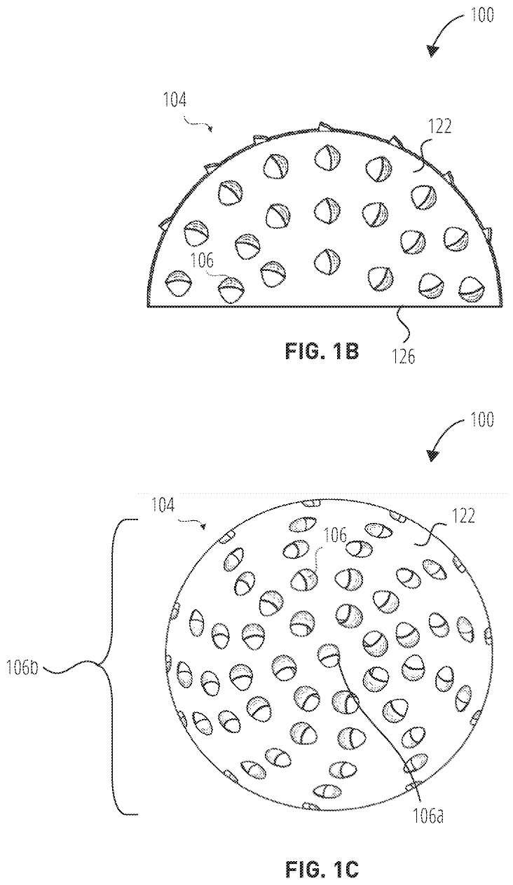

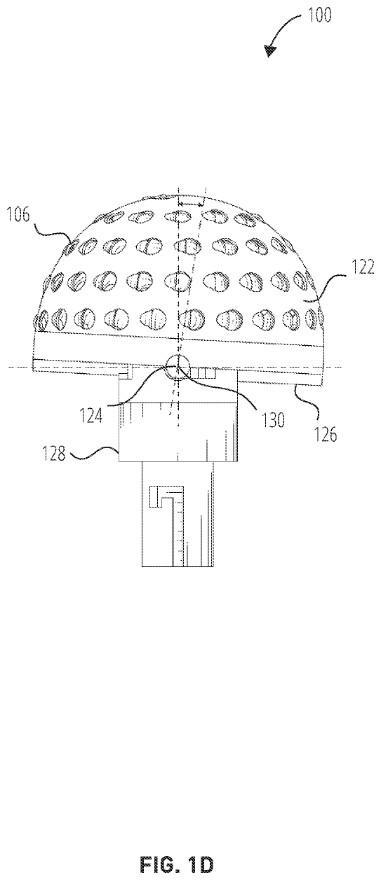

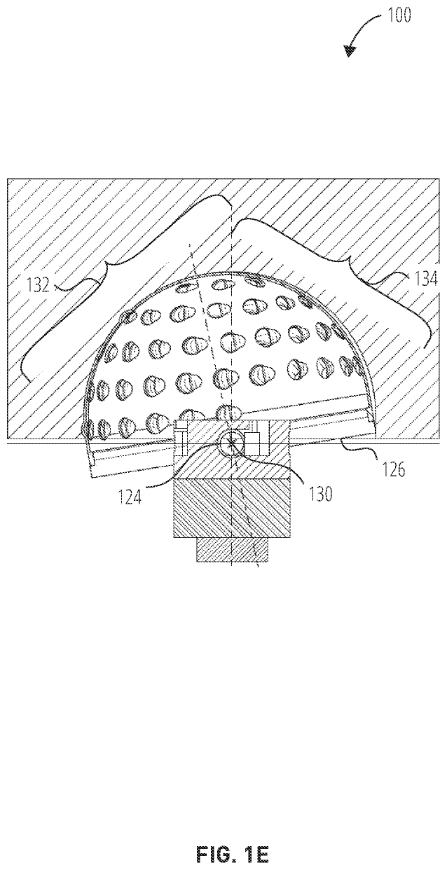

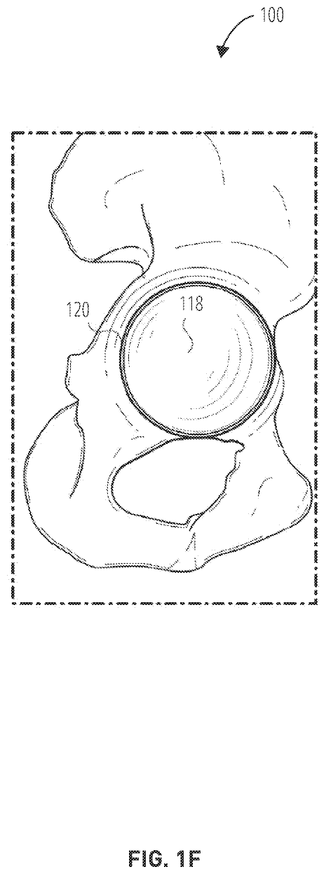

FIGS. 1A-1F are diagrams of an example 100 associated with an improved rotary tool. As shown in FIGS. 1A-1F, the example 100 includes an off-axis rotary tool 102 (e.g., an off-axis reamer). The off-axis rotary tool 102 includes a cutting head 104 (e.g., a reamer head). The cutting head 104 includes cutting teeth 106.

The cutting head 104 is driven via a motor drive 108 (e.g., the motor drive 108 may provide for rotational motion of the cutting head 104), as described in more detail elsewhere herein. The motor drive 108 may be any suitable drive mechanism that imparts at least rotational motion to the cutting head 104. During an operational cycle of the off-axis rotary tool 102, the cutting head 104 simultaneously precesses about a precession axis 110 and rotates about a rotation axis 112.

The simultaneous precession (e.g., about the precession axis 110 and in a direction shown by arrow 114 of FIG. 1A) and rotation (e.g., about the rotation axis 112 and in a direction indicated by arrow 116 in FIG. 1A) of the cutting head 104 enables the off-axis rotary tool 102 to create a cavity (e.g., a cavity 118 of an acetabulum 120, as shown in FIG. 1F) that does not contain excess material (e.g., bone material) while also significantly reducing an amount of linear force required for cutting (e.g., by significantly reducing a frictional load in the surgical process).

In some implementations, the cutting teeth 106 are provided on a surface 122 (e.g., a curved surface, such as a semi-hemispherical surface as shown in FIGS. 1A-1E,) of the cutting head 104 such that the cutting teeth 106 traverse across a center of rotation 124 during the operational cycle (e.g., when the cutting head 104 is simultaneously precessing and rotating, as described in more detail elsewhere herein). This enables the cutting head 104 to cut and remove material such that the off-axis rotary tool 102 may advance medially during the surgical process. This is in contrast to typical rotary tools that are impeded from advancing medially because the typical rotary tools cannot cut and remove material at the same time.

In some implementations, the cutting teeth 106 are disposed proximate to a center of the rotation axis 112, which assists with cutting and removal of material in a center of a cavity (e.g., cutting and removal of material in a center of the cavity 118 of the acetabulum 120, as shown in FIG. 1F). Accordingly, for example, the off-axis rotary tool 102 may be used to cut and remove material from the center of rotation 124 of the precession axis 110.

As shown in FIG. 1C, the cutting teeth 106 include a central cutting tooth 106a and a set of cutting teeth rings 106b that are concentric relative to the central cutting tooth 106a (e.g., the set of concentric rings 106b surround the central cutting tooth 106a). Although particular configurations of the cutting teeth 106 are described herein, the cutting teeth 106 may take on any suitable configuration (e.g., the cutting teeth 106 may include any suitable number of cutting teeth and may be located at any suitable location relative to the cutting head 104).

The precession axis 110 and the rotation axis 112 may extend from the center of rotation 124 of the cutting head 104. As an example, and as shown in FIG. 1A, the center of rotation 124 is located a distance away from a rim 126 of the cutting head 104. As another example, and as shown in FIGS. 1D-1E, the center of rotation 124 is located at a point that is located on the rim 126 of the cutting head 104 (e.g., an adapter 128 allows precessional motion about a theoretical center of rotation). Although the center of rotation 124 is shown and described as being in located in particular locations, the center of rotation 124 may be located in any suitable location.

As further shown in FIGS. 1D-1E, the center of rotation 124 is located at a center point 130 of the cutting head 104. Because the center of rotation 124 is located at the center point 130 of the cutting head 104, the off-axis rotary tool 102 may be used to create a cavity (e.g., the cavity 118 of the acetabulum 120, as shown in FIG. 1F) having a size that is based on a size of the cutting head 104. In this way, the size of the cavity may be equal to a specification size of the cutting head 104 (e.g., the cutting head size may be equal to specified reamer cup sizes, among other examples).

In some implementations, the precession axis 110 extends (or passes) through a center of an area that is intended to be operated on and the rotation axis 112 extends (or passes) through a center of the cutting head such that a predetermined angle between the precession axis 110 and the rotation axis 112 is created. In other words, the precession axis 110 and the rotation axis 112 may be offset from one another by an angle, relative to the center of rotation 124, to define an intended cutting area of the cutting head 104, as described in more detail elsewhere herein.

As an example, an angle, relative to the center of rotation 124, between the precession axis 110 and the rotation axis 112 may be in a range of approximately 3 degrees to 6 degrees. Although the angle, relative to the center of rotation 124, between the precession axis 110 and the rotation axis 112 is described as being in the range of approximately 3 degrees to 6 degrees, the angle, relative to the center of rotation 124, between the precession axis 110 and the rotation axis 112 may be any suitable angle.

In some implementations, the simultaneous precession of the cutting head 104 about the precession axis 110 and the rotation of the cutting head 104 about the rotation axis 112 enables only a portion of the cutting teeth 106 to be in contact with a cutting surface (e.g., the cavity 118 of the acetabulum 120, as shown in FIG. 1F) at any given time during a surgical process (e.g., a reaming process). As shown in FIG. 1E, a first set of cutting teeth 132 are in contact with the acetabulum while a second set of cutting teeth 134 are not in contact with the acetabulum at a particular point in time during the operational cycle of the off-axis rotary tool 102.

As another example, if the cutting head 104 has 40 teeth, only 8 teeth may ever come into contact with the cutting surface at any given time during the surgical process (e.g., only 8 teeth are within the intended cutting area during the precession and the rotation of the cutting head 104). As a result, the linear force required to cut the cutting surface and the frictional load associated with the surgical process is reduced (e.g., the linear force required to cut the cutting surface and the frictional load associated with the surgical process are reduced by 80%). It should be appreciated that these numbers are exemplary as the cutting head 104 may include any suitable number of cutting teeth. Additionally, or alternatively, the angle of the rotation axis 112 may be changed to cause more or less cutting teeth to come into contact with the cutting surface (e.g., within the intended cutting area as the cutting head 104 simultaneously precesses and rotates).

Accordingly, some implementations described herein may provide a solution to high reactionary forces that occur from the use of orthopedic surgical tools. In this way, the reactionary forces upon a surgical robot and/or a surgeon are reduced, allowing better control of the orthopedic surgical tool (e.g., positioning thereof) during surgery. In addition to reducing the reactionary forces, some implementations described herein mechanically provide all, or a significant portion of, linear forces required to complete surgeries (e.g., orthopedic surgeries). In this way, the surgeon and/or the surgical robot only have to guide the orthopedic surgical tools while imparting minimal force onto the orthopedic surgical tools.

Additionally, some implementations described herein enable an off-axis rotary tool (e.g., an off-axis reamer) to create a cavity that does not contain excess material while also significantly reducing an amount of force required for cutting during an orthopedic procedure (e.g., by significantly reducing a frictional load associated with the surgical process), as described in more detail elsewhere herein. Furthermore, some implementations described herein enable the off-axis rotary tool to provide sufficient linear force to move (or advance) the off-axis rotary tool medially, as described in more detail elsewhere herein.

As indicated above, FIGS. 1A-1F are provided as an example. Other examples may differ from what is described with regard to FIGS. 1A-1F.

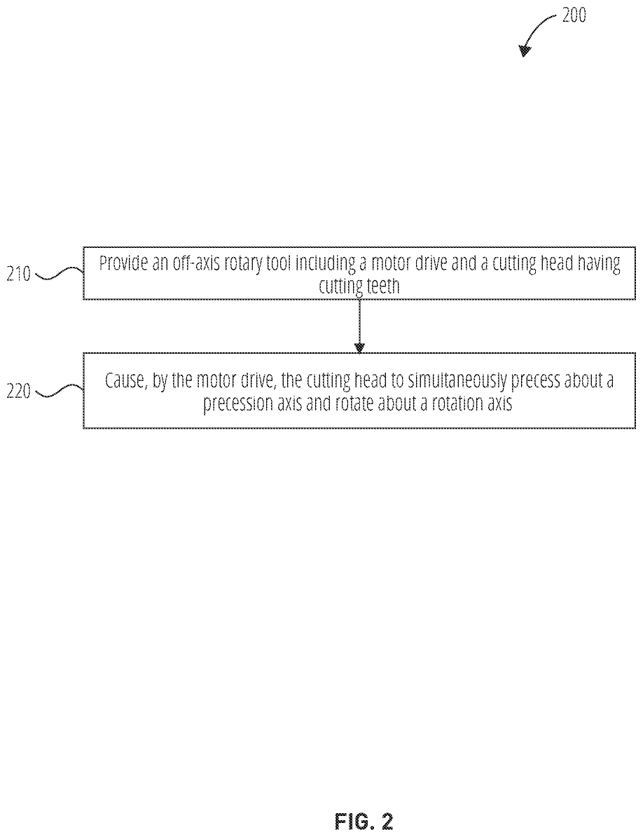

FIG. 2 is a flowchart of an example process 200 associated with an improved rotary tool. As shown in FIG. 2, the process 200 may include providing an off-axis rotary tool (e.g., the off-axis rotary tool 102) including a motor drive (e.g., the motor drive 108) and cutting head (e.g., the cutting head 104) having cutting teeth (e.g., the cutting teeth 106) (block 210), as described in more detail elsewhere herein. The cutting head may be driven via the motor drive.

As shown in FIG. 2, the process 200 includes causing, by the motor drive, the cutting head to simultaneously precess about a precession axis (e.g., the precession axis 110) and rotate about a rotation axis (e.g., the rotation axis 112) (block 220), as described in more detail elsewhere herein. In some implementations, the precession axis and the rotation axis may extend from a center of rotation (e.g., the center of rotation 124) of the cutting head such that the precession axis and the rotation axis are offset from one another by an angle, relative to the center of rotation, to define an intended cutting area of the cutting head. During an operational cycle of the off-axis rotary tool, the cutting teeth pass through the intended cutting area such that only a portion of the cutting teeth are located within the intended cutting area at any given time. In other words, as the cutting head precesses about the precession axis and rotates about the rotation axis, only a portion of the cutting teeth are within the intended cutting area at any point in time during the operational cycle.

In some implementations, the cutting teeth may be located at positions that are more aligned with the rotation axis than the precession axis. In some implementations, the precession axis and the rotation axis extend from the center of rotation and the angle between the precession axis and the rotation axis, relative to the center of rotation, is in a range of approximately 3 degrees to 6 degrees.

In some implementations, the cutting teeth may include a central cutting tooth (e.g., the central cutting tooth 106a) and a set of cutting teeth rings (e.g., the set of concentric rings 106b) that are concentric relative to the central cutting tooth. The cutting teeth may be provided on a surface (e.g., the surface 122) of the cutting head and the cutting head may simultaneously precess about the precession axis and rotate about the rotation axis from the center of rotation. A distance from the center of rotation to points (e.g., all points) on the surface may be any suitable distance (e.g., uniform or not uniform). In some implementations, the cutting head may be a semi-hemispherical structure, such as a semi-hemispherical reamer cup.

Although FIG. 2 shows example blocks of process 200, in some implementations, process 200 may include additional blocks, fewer blocks, different blocks, or differently arranged blocks than those depicted in FIG. 2. Additionally, or alternatively, two or more of the blocks of process 200 may be performed in parallel. The process 200 is an example of one process that may be performed by one or more devices described herein. These one or more devices may perform one or more other processes based on operations described herein, such as the operations described in connection with FIGS. 1A-1F. Moreover, while the process 200 has been described in relation to the devices and components of the preceding figures, the process 200 can be performed using alternative, additional, or fewer devices and/or components. Thus, the process 200 is not limited to being performed with the example devices, components, hardware, and software explicitly enumerated in the preceding figures.

The description set forth herein in connection with the appended drawings is intended as a description of various embodiments of the disclosed subject matter and is not necessarily intended to represent the only embodiment(s). In certain instances, the description includes specific details for the purpose of providing an understanding of the disclosed subject matter. However, it will be apparent to those skilled in the art that embodiments may be practiced without these specific details. In some instances, well-known structures and components may be shown in block diagram form to avoid obscuring the concepts of the disclosed subject matter.

Reference throughout the specification to “one embodiment” or “an embodiment” means that a particular feature, structure, characteristic, operation, or function described in connection with an embodiment is included in at least one embodiment of the disclosed subject matter. Thus, any appearance of the phrases “in one embodiment” or “in an embodiment” in the specification is not necessarily referring to the same embodiment. Further, the particular features, structures, characteristics, operations, or functions may be combined in any suitable manner in one or more embodiments. Further, it is intended that embodiments of the disclosed subject matter can and do cover modifications and variations of the described embodiments.

It must be noted that, as used in the specification and the appended claims, the singular forms “a,” “an,” and “the” include plural referents unless the context clearly dictates otherwise. That is, unless clearly specified otherwise, as used herein the words “a” and “an” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein, merely describe points of reference and do not necessarily limit embodiments of the disclosed subject matter to any particular orientation or configuration. Furthermore, terms such as “first,” “second,” “third,” etc., merely identify one of a number of portions, components, points of reference, operations and/or functions as described herein, and likewise do not necessarily limit embodiments of the disclosed subject matter to any particular configuration or orientation.

Having described embodiments of the disclosed subject matter, it should be apparent to those skilled in the art that the foregoing is merely illustrative and not limiting, having been presented by way of example only. Thus, although particular configurations have been discussed herein, other configurations can also be employed. Numerous modifications and other embodiments (e.g., combinations, rearrangements, etc.) are enabled by the present disclosure and are within the scope of one of ordinary skill in the art and are contemplated as falling within the scope of the disclosed subject matter and any equivalents thereto. Features of the disclosed embodiments can be combined, rearranged, omitted, etc., within the scope of the invention to produce additional embodiments. Furthermore, certain features may sometimes be used to advantage without a corresponding use of other features. Accordingly, Applicant(s) intend(s) to embrace all such alternatives, modifications, equivalents, and variations that are within the spirit and scope of the disclosed subject matter.

What is claimed is:

1. An off-axis rotary tool, comprising:
a motor drive; and
a cutting head, coupled to the motor drive, including cutting teeth,
wherein, during an operational cycle of the off-axis rotary tool, the cutting head simultaneously precesses about a precession axis and rotates about a rotation axis.

2. The off-axis rotary tool of claim 1, wherein the precession axis and the rotation axis extend from a center of rotation of the cutting head,
wherein the precession axis and the rotation axis are offset from one another by an angle, relative to the center of rotation, to define an intended cutting area of the cutting head, and
wherein, during the operational cycle, the cutting teeth pass through the intended cutting area such that only a portion of the cutting teeth are located within the intended cutting area.

3. The off-axis rotary tool of claim 1, wherein the cutting teeth are located at positions that are more aligned with the rotation axis than the precession axis.

4. The off-axis rotary tool of claim 1, wherein the precession axis and the rotation axis extend from a center of rotation, and
wherein an angle between the precession axis and the rotation axis, relative to the center of rotation, is in a range of approximately 3 degrees to 6 degrees.

5. The off-axis rotary tool of claim 1, wherein the cutting teeth include a central cutting tooth and a set of concentric cutting teeth rings that surround the central cutting tooth.

6. The off-axis rotary tool of claim 1, wherein the cutting teeth are provided on a surface of the cutting head,
wherein the cutting head simultaneously precesses about the precession axis and rotates about the rotation axis from a center of rotation, and
wherein a distance from the center of rotation to all points on the surface is not uniform.

7. The off-axis rotary tool of claim 1, wherein the cutting head is a semi-hemispherical reamer cup.

8. A method of operating an off-axis rotary tool, the off-axis rotary tool including a motor drive and a cutting head, coupled to the motor drive, including cutting teeth, the method comprising:
causing, via the motor drive, the cutting head to simultaneously precess about a precession axis and rotate about a rotation axis.

9. The method of claim 8, wherein the precession axis and the rotation axis extend from a center of rotation of the cutting head,
wherein the precession axis and the rotation axis are offset from one another by an angle, relative to the center of rotation, to define an intended cutting area of the cutting head, and
wherein, during an operational cycle of the off-axis rotary tool, the cutting teeth pass through the intended cutting area such that only a portion of the cutting teeth are located within the intended cutting area.

10. The method of claim 8, wherein the cutting teeth are located at positions that are more aligned with the rotation axis than the precession axis.

11. The method of claim 8, wherein the precession axis and the rotation axis extend from a center of rotation, and
wherein an angle between the precession axis and the rotation axis, relative to the center of rotation, is in a range of approximately 3 degrees to 6 degrees.

12. The method of claim 8, wherein the cutting teeth include a central cutting tooth and a set of cutting teeth rings that are concentric relative to the central cutting tooth.

13. The method of claim 8, wherein the cutting head is a semi-hemispherical reamer cup.

14. The method of claim 8, wherein the cutting teeth are provided on a surface of the cutting head,
wherein the cutting head simultaneously precesses about the precession axis and rotates about the rotation axis from a center of rotation, and
wherein a distance from the center of rotation to all points on the surface is not uniform.

15. An off-axis reamer for orthopedic surgery, the off-axis reamer comprising:
a motor drive; and
a reamer head, coupled to the motor drive, including cutting teeth,
wherein, during an operational cycle of the off-axis reamer, the reamer head simultaneously precesses about a precession axis and rotates about a rotation axis.

16. The off-axis reamer of claim 15, wherein the reamer head includes a semi-hemispherical curved surface,
wherein the reamer head precesses about the precession axis and rotates about the rotation axis from a center of rotation, and
wherein a distance from the center of rotation to all points on the semi-hemispherical curved surface is uniform.

17. The off-axis reamer of claim 15, wherein the rotation axis extends from a center of rotation through a center of the reamer head, and
wherein the precession axis extends from the center of rotation through a portion of the reamer head that is based on an angle, relative to the center of rotation, between the rotation axis and the precession axis.

18. The off-axis reamer of claim 15, wherein the precession axis and the rotation axis extend from a center of rotation, and
wherein an angle, relative to the center of rotation, between the precession axis and the rotation axis is in a range of approximately 3 degrees to 6 degrees.

19. The off-axis reamer of claim 15, wherein the cutting teeth are located at positions that are more aligned with the rotation axis than the precession axis.

20. The off-axis reamer of claim 15, wherein the cutting teeth include a central cutting tooth and a set of concentric cutting teeth rings that surround the central cutting tooth.
